(19) United States
(12) Patent Application Publication
(10) Pub. No.: US 2026/0092911 A1
(43) Pub. Date: Apr. 2, 2026

(54) IN-SITU HEAVY METAL MEASUREMENT SYSTEM AND METHOD BASED ON ASV THAT ACCOUNTS FOR INTERFERENCE FROM ENVIRONMENTAL FACTORS IN GROUNDWATER

(71) Applicant: Institute of Soil Science, Chinese Academy of Sciences, Nanjing (CN)

(72) Inventors: Xicai PAN, Nanjing (CN); Yusong LIU, Nanjing (CN); Jiabao ZHANG, Nanjing (CN); Lei QIAO, Nanjing (CN); Li MIAO, Nanjing (CN); Hua ZHANG, Guiyang (CN)

(73) Assignee: Institute of Soil Science, Chinese Academy of Sciences, Nanjing (CN)

(21) Appl. No.: 19/335,975

(22) Filed: Sep. 22, 2025

(30) Foreign Application Priority Data
Sep. 30, 2024 (CN) 202411380734.7

(51) Int. Cl. G01N 33/18

(52) U.S. Cl. CPC G01N 33/1813

(57) ABSTRACT

An in-situ heavy metal measurement system based on ASV that accounts for interference from environmental factors in groundwater includes: an ASV sensor measurement module, an environmental factor data collection module, a data transmission module, an adaptive intelligent correction algorithm module, and a data output module. The environmental factor data collection module collects pH and the temperature data of groundwater near a measurement location of a heavy metal when the ASV sensor measurement module performs in-situ measurement, the pH and the temperature obtained by the environmental factor data collection module and a measurement result of the ASV sensor measurement module are used as inputs of a adaptive intelligent correction algorithm model, and a corrected measurement result is predicted by the adaptive intelligent correction algorithm model to obtain a corrected result, thus the accuracy and reliability of ASV-based heavy metal measurement in groundwater can be significantly improved.

### CROSS-REFERENCE TO RELATED APPLICATIONS

This application claims priority to Chinese Patent Application No. 202411380734.7, filed on Sep. 30, 2024, which is herein incorporated by reference in its entirety.

### TECHNICAL FIELD

The disclosure relates to the field of in-situ heavy metal measurement technologies, and more particularly to an in-situ heavy metal measurement system and method based on anodic stripping voltammetry (ASV) that accounts for interference from environmental factors in groundwater.

### BACKGROUND

ASV, based on electrochemical principles, enables quantitative analysis of heavy metal ions through precise control of electrode potential and current. This method offers high sensitivity, a low measurement limit, and a capability for simultaneous multi-parameter measurement. Furthermore, ASV is operationally straightforward, and the entire measurement process can typically be completed within a relatively short period. It is commonly and widely used for rapid measurement and emergency response in surface water/industrial wastewater. However, because ASV involves multiple reaction mechanisms and influencing factors within the electrochemical process, the reproducibility of measurement results is relatively poor. In practical applications, it is necessary not only to strictly control experimental conditions, such as electrode state, enrichment time, stirring speed, among numerous other parameters, but also to minimize the influence of interfering factors in the water environment.

Conventional ASV equipment measurement processes typically include equipment calibration and target measurement. During the equipment calibration, a standard curve model is established using measurement results from a blank sample and standard samples of specific concentrations. This model is subsequently employed to predict heavy metal concentrations of target samples. During in-situ or field measurement of heavy metals in groundwater, the temperature difference between the standard samples used in above-ground calibration and the groundwater to be measured can exceed 10° C. It is well-known that the temperature difference can affect electrode reaction rates and ion diffusion rates, thereby influencing the ASV measurement results. However, this interfering factor is frequently overlooked in current ASV-based groundwater measurement processes. Furthermore, supporting electrolytes and buffer solutions added during the ASV measurement process are typically of a fixed volume. However, the potential of hydrogen (pH) of the groundwater at contaminated sites often exhibits a wide variation range. The added electrolytes and buffer solutions may be insufficient to significantly eliminate interference from coexisting ions, leading to inaccurate measurement results.

### SUMMARY

In view of the above-described deficiencies in the related art, an in-situ heavy metal measurement system and method based on ASV that accounts for interference from environmental factors in groundwater, as provided by the disclosure, solves the problem of inaccurate measurement results caused by existing ASV measurement methods failing to consider the influence of temperature and pH on heavy metal measurements.

In order to achieve the above purposes, the technical solutions adopted by the disclosure are as follows.

An in-situ heavy metal measurement system based on ASV that accounts for interference from environmental factors in groundwater, includes: an ASV sensor measurement module, an environmental factor data collection module, a data transmission module, an adaptive intelligent correction algorithm module (also referred to adaptive correction algorithm module), and a data output module.

The ASV sensor measurement module includes an ASV sensor, and the ASV sensor measurement module is configured to perform in-situ measurement on a heavy metal in the groundwater to obtain an in-situ heavy metal measurement result.

The environmental factor data collection module includes a pH sensor and a temperature sensor; the environmental factor data collection module is configured to collect pH and temperature data of the groundwater near a measurement location of the heavy metal when the ASV sensor measurement module performs the in-situ measurement on the heavy metal in the groundwater.

The data transmission module is configured to transmit the in-situ heavy metal measurement result obtained by the ASV sensor measurement module, and the pH and temperature data obtained by the environmental factor data collection module to the adaptive intelligent correction algorithm module.

The adaptive intelligent correction algorithm module includes an adaptive intelligent correction algorithm model (also referred to as adaptive correction algorithm model), and the adaptive intelligent correction algorithm module is configured to take the in-situ heavy metal measurement result obtained by the ASV sensor measurement module and the pH and temperature data obtained by the environmental factor data collection module as inputs to predict a corrected measurement result, thereby obtaining a corrected result.

The data output module is configured to output the corrected result obtained by the adaptive intelligent correction algorithm module as a final measurement result for the heavy metal in the groundwater.

In an embodiment, the data transmission module includes a data transmission connector, the adaptive intelligent correction algorithm module includes a processor, and the data output module includes a data output connector.

In an embodiment, the final measurement result for the heavy metal is outputted to a display for visualization; in addition, the environmental remediation company performs groundwater remediation based on the final measurement result for the heavy metal.

In an embodiment, a specific method for taking the in-situ heavy metal measurement result obtained by the ASV sensor measurement module and the pH and temperature data obtained by the environmental factor data collection module as the inputs to predict the corrected measurement result, includes:
taking the in-situ heavy metal measurement result obtained by the ASV sensor measurement module and the pH and temperature data of the groundwater near the measurement location obtained by the environmental factor data collection module as the inputs to a trained adaptive intelligent correction algorithm model; and
taking an output of the trained adaptive intelligent correction algorithm model as the corrected result, thereby completing correction of the in-situ heavy metal measurement result from the ASV sensor measurement module; the adaptive intelligent correction algorithm model is a multi-layer perceptron (MLP) regression model.

In an embodiment, a training method for the adaptive intelligent correction algorithm model includes:
based on measurement ranges of the ASV sensor, selecting detection scopes of each measurement range in a laboratory to measure samples with known heavy metal concentration values under different temperature and pH values to obtain multiple sets of sample measurement data;
combining the sample measurement data, the actual heavy metal concentration value (also referred to known heavy metal concentration value), the temperature value and the pH value under the same environment condition into a piece of training data, thereby obtaining multiple training data sets corresponding to different measurement ranges;
inputting the multiple training data sets corresponding to the different measurement ranges into the adaptive intelligent correction algorithm model to predict the corrected measurement result and output the corrected result; and
calculating a loss value based on actual groundwater heavy metal data and the corrected result, and optimizing the adaptive intelligent correction algorithm model via backpropagation until a training requirement of the adaptive intelligent correction algorithm model is met, thereby obtaining the trained adaptive intelligent correction algorithm model.

In an embodiment, a specific method for the selecting detection scopes of each measurement range in a laboratory to measure samples with known heavy metal concentration values under different temperature and pH values to obtain multiple sets of sample measurement data, includes:
preparing high-concentration standard solutions and low-concentration standard solutions in the detection scopes of heavy metal to be measured of a selected measurement range;
based on the high-concentration standard solutions and the low-concentration standard solutions, preparing m samples each with the known heavy metal concentration values across the pH values ranging from 1 to 12; and
placing the m samples in a temperature control box, sequentially setting n control temperatures uniformly distributed across a conventional groundwater temperature range, and measuring a heavy metal concentration value of each sample at each control temperature according to a standard measurement process of the ASV sensor, thereby obtaining the multiple sets of sample measurement data.

In an embodiment, a concentration value C_{H} of the high-concentration standard solutions is calculated as:

$${C}_{H} = {C}_{min} + 0.8 \times ( {C}_{max} - {C}_{min} )$$

where Cₘᵢₙ is a lower limit of the detection scopes of the selected measurement range, and Cₘₐₓ is an upper limit of the detection scopes of the selected measurement range.

In an embodiment, a concentration value C_{L} of the low-concentration standard solutions is calculated as:

$${C}_{L} = {C}_{min} + 0.1 \times ( {C}_{max} - {C}_{min} )$$

where Cₘᵢₙ is a lower limit of the detection scopes of the selected measurement range, and Cₘₐₓ is an upper limit of the detection scopes of the selected measurement range.

In an embodiment, m and n are each in a range of 5 to 10, and the conventional groundwater temperature range is 5° C. to 35° C.

An in-situ heavy metal measurement method based on ASV that accounts for interference from environmental factors in groundwater, implemented by the above in-situ heavy metal measurement system, includes:
S1: performing the in-situ measurement on the heavy metal in the groundwater via the ASV sensor measurement module;
S2: collecting the pH and temperature data of the groundwater near the measurement location of the heavy metal via the environmental factor data collection module when the ASV sensor measurement module performs the in-situ measurement on the heavy metal in the groundwater;
S3: transmitting the in-situ heavy metal measurement result obtained by the ASV sensor measurement module and the pH and temperature data obtained by the environmental factor data collection module to the adaptive intelligent correction algorithm module via the data transmission module;
S4: inputting the pH and temperature data obtained by the environmental factor data collection module and the in-situ heavy metal measurement result obtained by the ASV sensor measurement module into the adaptive intelligent correction algorithm model via the adaptive intelligent correction algorithm module, predicting the corrected measurement result via the adaptive intelligent correction algorithm model, thereby obtaining the corrected result; and
S5: outputting the corrected result obtained by the adaptive intelligent correction algorithm module as the final measurement result for the heavy metal in the groundwater via the data output module.

The beneficial effects of the disclosure are as follows: the environmental factor data collection module collects the pH and temperature data of the groundwater near a measurement location of the heavy metal when the ASV sensor measurement module performs the in-situ measurement, the pH and temperature data obtained by the environmental factor data collection module and the measurement result obtained by the ASV sensor measurement module are used as inputs of the adaptive intelligent correction algorithm model. Through this model, the corrected measurement result is predicted, yielding the final corrected result. The disclosure can significantly enhance the accuracy and reliability of ASV-based heavy metal measurement in groundwater.

### BRIEF DESCRIPTION OF DRAWINGS

FIG. 1 illustrates a schematic structural diagram of an in-situ heavy metal measurement system according to an embodiment of the disclosure.

FIG. 2 illustrates performance of an adaptive intelligent correction algorithm model on training data sets and measurement data for measurement of heavy metal Pb using ASV within a range of 0-100 ppb according to an embodiment of the disclosure; wherein:
(a) shows a comparison of error before and after correction under different pH conditions at an actual concentration of 10 ppb in the training set;
(b) shows a comparison of error before and after correction under different temperature conditions at an actual concentration of 10 ppb in the training set;
(c) shows a comparison of error before and after correction under different pH conditions at an actual concentration of 80 ppb in the training set;
(d) shows a comparison of error before and after correction under different temperature conditions at an actual concentration of 80 ppb in the training set;
(e) shows a comparison of measurement error before and after correction for both the training set and the validation set.

### DETAILED DESCRIPTION OF THE EMBODIMENTS

Specific embodiments of the disclosure are described below, so that those skilled in the art can understand the disclosure. However, it should be clear that the disclosure is not limited to the scope of specific embodiments. As long as various changes are within the spirit and scope of the disclosure as defined and determined by the appended claims, all inventions and creations using the concept of the disclosure are protected.

### Embodiment 1

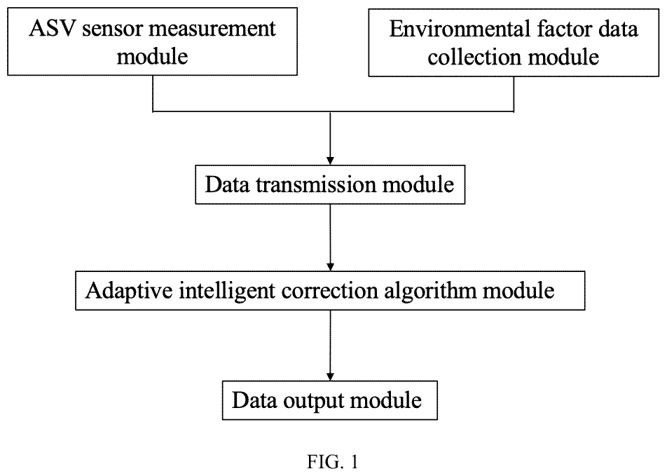

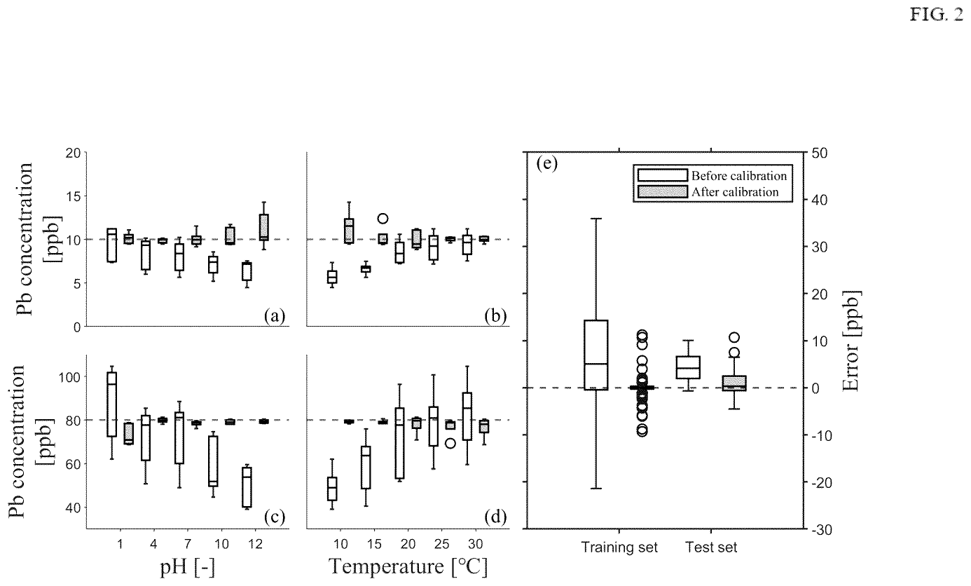

As shown in FIG. 1, an in-situ heavy metal measurement system based on ASV that accounts for interference from environmental factors in groundwater, includes: an ASV sensor measurement module, an environmental factor data collection module, a data transmission module, an adaptive intelligent correction algorithm module, and a data output module.

The ASV sensor measurement module includes an ASV sensor, and the ASV sensor measurement module is configured to perform in-situ measurement on a heavy metal in the groundwater to obtain an in-situ heavy metal measurement result.

The environmental factor data collection module includes a pH sensor and a temperature sensor; and the environmental factor data collection module is configured to collect pH and temperature data of the groundwater near a measurement location of the heavy metal when the ASV sensor measurement module performs the in-situ measurement on the heavy metal in the groundwater.

The data transmission module is configured to transmit the in-situ heavy metal measurement result obtained by the ASV sensor measurement module, and the pH and temperature data obtained by the environmental factor data collection module to an adaptive intelligent correction algorithm module.

The adaptive intelligent correction algorithm module includes an adaptive intelligent correction algorithm model, and the adaptive intelligent correction algorithm module is configured to take the in-situ heavy metal measurement result obtained by the ASV sensor measurement module and the pH and temperature data obtained by the environmental factor data collection module as inputs to predict a corrected measurement result, thereby obtaining a corrected result.

The data output module is configured to output the corrected result obtained by the adaptive intelligent correction algorithm module as a final measurement result for the heavy metal in the groundwater.

A specific method for taking the in-situ heavy metal measurement result obtained by the ASV sensor measurement module and the pH and temperature data obtained by the environmental factor data collection module as the inputs to predict the corrected measurement result includes:
taking the in-situ heavy metal measurement result obtained by the ASV sensor measurement module and the pH and temperature data of the groundwater near the measurement location obtained by the environmental factor data collection module as the inputs to a trained adaptive intelligent correction algorithm model; and
taking an output of the trained adaptive intelligent correction algorithm model as the corrected result, thereby completing correction of the in-situ heavy metal measurement result from the ASV sensor measurement module.

The adaptive intelligent correction algorithm model is a MLP regression model.

A training method for the adaptive intelligent correction algorithm model includes:
based on measurement ranges of the ASV sensor, selecting detection scopes of each measurement range in a laboratory to measure samples with known heavy metal concentration values under different temperature and pH values to obtain multiple sets of sample measurement data;
obtaining multiple training data sets corresponding to the measurement ranges by taking the multiple sets of sample measurement data, actual heavy metal concentration values, the temperature and pH values collected under same environment conditions as training data;
inputting the multiple training data sets corresponding to the measurement ranges into the adaptive intelligent correction algorithm model to predict the corrected measurement result and output the corrected result; and
calculating a loss value based on actual groundwater heavy metal data and the corrected result, and optimizing the adaptive intelligent correction algorithm model via backpropagation until a training requirement of the adaptive intelligent correction algorithm model is met, thereby obtaining the trained adaptive intelligent correction algorithm model.

A specific method for the selecting detection scopes of each measurement range in a laboratory to measure samples with known heavy metal concentration values under different temperature and pH values to obtain multiple sets of sample measurement data, includes:
preparing high-concentration standard solutions and low-concentration standard solutions in heavy metal detection scopes to be measured of a selected measurement range;
based on the high-concentration standard solutions and the low-concentration standard solutions, preparing m samples each with the known heavy metal concentration values across the pH values ranging from 1 to 12; and
placing all samples in a temperature control box, sequentially setting n control temperatures uniformly distributed across a conventional groundwater temperature range, and measuring a heavy metal concentration value of each sample at each control temperature according to a standard measurement process of the ASV sensor, thereby obtaining the multiple sets of sample measurement data.

A concentration value C_{H} of the high-concentration standard solutions is calculated as:

$${C}_{H} = {C}_{min} + 0.8 \times ( {C}_{max} - {C}_{min} )$$

where Cₘᵢₙ is a lower limit of the detection scopes of the selected measurement range, and Cₘₐₓ is an upper limit of the detection scopes of the selected measurement range.

A concentration value C_{L} of the low-concentration standard solutions is calculated as:

$${C}_{L} = {C}_{min} + 0.1 \times ( {C}_{max} - {C}_{min} )$$

where Cₘᵢₙ is a lower limit of the detection scopes of the selected measurement range, and Cₘₐₓ is an upper limit of the detection scopes of the selected measurement range.

In an embodiment, m and n are each in a range of 5 to 10, and the conventional groundwater temperature range is 5° C. to 35° C.

### Embodiment 2

This embodiment is a further extension of Embodiment 1. In this embodiment, the heavy metal in the groundwater is lead element (Pb), and the high-concentration standard solution and low-concentration standard solution set in the training set are 80 ppb and 10 ppb respectively, and the values of m and n corresponding to the pH and temperature of the training set are both 5; the pH is 1, 5, 7, 10 and 12 respectively, and the temperature is 10, 15, 20, 25 and 30° C. respectively. The verification set includes 50 simulated actual groundwater samples.

An in-situ heavy metal measurement method based on ASV that accounts for interference from environmental factors in groundwater includes the following steps S0 to S5.
S0, the adaptive intelligent correction algorithm is trained based on the above training set.
S1, the in-situ measurement of the heavy metal in the validation set is carried out through the ASV sensor measurement module.
S2, when the heavy metal in the verification set is measured in situ by the ASV sensor measurement module, and the pH and temperature data near the measurement location are collected through the environmental factor data collection module.
S3, the data obtained by the ASV sensor measurement module and the environmental factor data collection module are transmitted to the adaptive intelligent correction algorithm module through the data transmission module.
S4, the data obtained by the environmental factor data collection module and the measurement result obtained by the ASV sensor measurement module are used as the inputs of the adaptive intelligent correction algorithm model, and the corrected measurement result is predicted by the adaptive intelligent correction algorithm model to obtain the corrected result.
S5, through the data output module, the corrected result obtained by the adaptive intelligent correction algorithm module is used as the final heavy metal measurement result in the verification set and output.

In this embodiment, the root mean square error (RMSE) of the ASV measured value of the actual groundwater sample in the verification set is 9.65 ppb. As shown in FIG. 2, after correction by the method, the RMSE is reduced to 5.8 ppb; the average error before correction is 4.21 ppb, and the average error after correction is 1.69 ppb. Therefore, the error is reduced after correction by this method, and the accuracy and reliability of ASV-based heavy metal measurement in groundwater are significantly improved.

In summary, the environmental factor data collection module collects the pH and the temperature data of groundwater nearby when the ASV sensor measurement module performs in-situ measurement, the data obtained by the environmental factor data collection module and the measurement result obtained by the ASV sensor measurement module are used as the inputs of the adaptive intelligent correction algorithm model, and the corrected measurement result is predicted by the adaptive intelligent correction algorithm model to obtain the corrected result, thus the accuracy and reliability of ASV-based heavy metal measurement in groundwater can be significantly improved.

What is claimed is:

1. An in-situ heavy metal measurement system based on anodic stripping voltammetry (ASV) that accounts for interference from environmental factors in groundwater, comprising:
an ASV sensor measurement module, comprising an ASV sensor, wherein the ASV sensor measurement module is configured to perform in-situ measurement on a heavy metal in the groundwater to obtain an in-situ heavy metal measurement result;
an environmental factor data collection module, comprising a potential of hydrogen (pH) sensor and a temperature sensor, wherein the environmental factor data collection module is configured to collect pH and temperature data of the groundwater near a measurement location of the heavy metal when the ASV sensor measurement module performs the in-situ measurement on the heavy metal in the groundwater;
a data transmission module, configured to transmit the in-situ heavy metal measurement result obtained by the ASV sensor measurement module, and the pH and temperature data obtained by the environmental factor data collection module to an adaptive intelligent correction algorithm module;
the adaptive intelligent correction algorithm module, comprising an adaptive intelligent correction algorithm model, wherein the adaptive intelligent correction algorithm module is configured to take the in-situ heavy metal measurement result obtained by the ASV sensor measurement module and the pH and temperature data obtained by the environmental factor data collection module as inputs to predict a corrected measurement result, thereby obtaining a corrected result; and
a data output module, configured to output the corrected result obtained by the adaptive intelligent correction algorithm module as a final measurement result for the heavy metal in the groundwater;
wherein a training method for the adaptive intelligent correction algorithm model, comprises:
based on measurement ranges of the ASV sensor, selecting detection scopes of each measurement range in a laboratory to measure samples with known heavy metal concentration values under different temperature and pH values to obtain multiple sets of sample measurement data;
obtaining a plurality of training data sets corresponding to the measurement ranges by taking the multiple sets of sample measurement data, actual heavy metal concentration values, the temperature and pH values collected under same environment conditions as training data;
inputting the plurality of training data sets corresponding to the measurement ranges into the adaptive intelligent correction algorithm model to predict the corrected measurement result and output the corrected result; and
calculating a loss value based on actual groundwater heavy metal data and the corrected result, and optimizing the adaptive intelligent correction algorithm model via backpropagation until a training requirement of the adaptive intelligent correction algorithm model is met, thereby obtaining a trained adaptive intelligent correction algorithm model;
wherein a specific method for taking the in-situ heavy metal measurement result obtained by the ASV sensor measurement module and the pH and temperature data obtained by the environmental factor data collection module as the inputs to predict the corrected measurement result, comprises:
taking the in-situ heavy metal measurement result obtained by the ASV sensor measurement module and the pH and temperature data of the groundwater near the measurement location obtained by the environmental factor data collection module as the inputs to the trained adaptive intelligent correction algorithm model; and
taking an output of the trained adaptive intelligent correction algorithm model as the corrected result, thereby completing correction of the in-situ heavy metal measurement result from the ASV sensor measurement module;
wherein the adaptive intelligent correction algorithm model is a multi-layer perceptron (MLP) regression model;
wherein a specific method for the selecting detection scopes of each measurement range in a laboratory to measure samples with known heavy metal concentration values under different temperature and pH values to obtain multiple sets of sample measurement data, comprises:
preparing high-concentration standard solutions and low-concentration standard solutions in the detection scopes of the heavy metal of a selected measurement range;
based on the high-concentration standard solutions and the low-concentration standard solutions, preparing m samples each with the known heavy metal concentration values across the pH values ranging from 1 to 12; and
placing the m samples in a temperature control box, sequentially setting n control temperatures uniformly distributed across a conventional groundwater temperature range, and measuring a heavy metal concentration value of each sample at each control temperature according to a standard measurement process of the ASV sensor, thereby obtaining the multiple sets of sample measurement data;
wherein m and n are each in a range of 5 to 10, and the conventional groundwater temperature range is 5° C. to 35° C.;
wherein a concentration value C_{H} of the high-concentration standard solutions is calculated as:
$${C}_{H} = {C}_{min} + 0.8 \times ( {C}_{max} - {C}_{min} )$$
where Cₘᵢₙ is a lower limit of the detection scopes of the selected measurement range, and Cₘₐₓ is an upper limit of the detection scopes of the selected measurement range;
wherein a concentration value C_{L} of the low-concentration standard solutions is calculated as:
$${C}_{L} = {C}_{min} + 0.1 \times ( {C}_{max} - {C}_{min} )$$
where Cₘᵢₙ is a lower limit of the detection scopes of the selected measurement range, and Cₘₐₓ is an upper limit of the detection scopes of the selected measurement range.

2. An in-situ heavy metal measurement method based on ASV that accounts for interference from environmental factors in groundwater, implemented by the in-situ heavy metal measurement system according to claim 1, comprising:
S1: performing the in-situ measurement on the heavy metal in the groundwater via the ASV sensor measurement module;
S2: collecting the pH and temperature data of the groundwater near the measurement location of the heavy metal via the environmental factor data collection module when the ASV sensor measurement module performs the in-situ measurement on the heavy metal in the groundwater;
S3: transmitting the in-situ heavy metal measurement result obtained by the ASV sensor measurement module and the pH and temperature data obtained by the environmental factor data collection module to the adaptive intelligent correction algorithm module via the data transmission module;
S4: inputting the pH and temperature data obtained by the environmental factor data collection module and the in-situ heavy metal measurement result obtained by the ASV sensor measurement module into the adaptive intelligent correction algorithm model via the adaptive intelligent correction algorithm module, predicting the corrected measurement result via the adaptive intelligent correction algorithm model, thereby obtaining the corrected result; and
S5: outputting the corrected result obtained by the adaptive intelligent correction algorithm module as the final measurement result for the heavy metal in the groundwater via the data output module.
